(19) United States
(12) Patent Application Publication
(10) Pub. No.: US 2023/0381083 A1
(43) Pub. Date: Nov. 30, 2023

(54) Phytic Acid Ester Derivative

(71) Applicant: NATIONAL UNIVERSITY CORPORATION KUMAMOTO UNIVERSITY, KUMAMOTO-SHI (JP)

(72) Inventors: Mikako Fujita, Kumamoto (JP); Masami Otsuka, Kumamoto (JP); Takeo Ohsugi, Hokkaido (JP); Hiroshi Tateishi, Kumamoto (JP); Naoki Murao, Kumamoto (JP); Takuya Masunaga, Kumamoto (JP)

(21) Appl. No.: 18/361,070

(22) Filed: Jul. 28, 2023

Related U.S. Application Data
(62) Division of application No. 17/186,664, filed on Feb. 26, 2021.
Application No. 17/186,664 is a continuation of application No. PCT/JP2019/034220, filed on Aug. 30, 2019.

(30) Foreign Application Priority Data
Aug. 30, 2018 (JP) 2018-161944

(51) Int. Cl. A61K 8/55; A61P 35/02; A61Q 19/02; C07F 9/09

(52) U.S. Cl. CPC A61K 8/55; A61P 35/02; A61Q 19/02; C07F 9/098

(57) ABSTRACT

The present invention relates to a phytic acid ester derivative and a use thereof. The phytic acid ester derivative of the present invention has a structure of the following formula I:

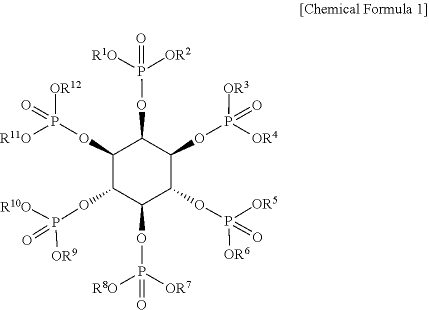

wherein R¹, R², R³, R⁴, R⁵, R⁶, R⁷, R⁸, R⁹, R¹⁰, R¹¹ and R¹² are each independently selected from the group consisting of H, the following formula II:

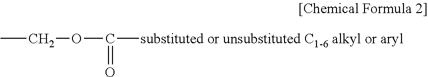

wherein —CH₂— is optionally substituted by one or two substituents, the following formula III:

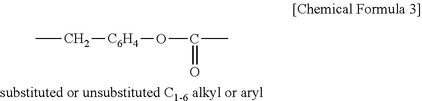

wherein —CH₂—C₆H₄— is optionally substituted by one or more substituents, and the following formula IV:

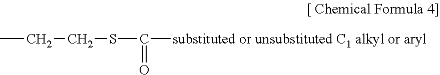

wherein —CH₂—CH₂— is optionally substituted by one or more substituents, provided that a compound where all of R¹ to R¹² are H is excluded.

### CROSS REFERENCE TO RELATED APPLICATIONS

The present application is a continuation application of International Application No. PCT/JP2019/034220, filed on Aug. 30, 2019.

### TECHNICAL FIELD

The present invention relates to a phytic acid ester derivative and a use thereof.

### BACKGROUND ART

Phytic acid (myo-inostiol-1,2,3,4,5,6-hexaphosphate, IP6) is a principal storage form of phosphorus biosynthesized in a cell of a mammal such as a human, and present also in a large number of plant tissues such as a seed. It is incepted also from food such as grains and beans. A part thereof is adsorbed, and is incorporated into a cell through pinocytosis or the like.

It has been well studied and noticed that addition of IP6 in a high concentration to a cancer cell exhibits a cell growth-inhibiting effect. Japanese Patent Laid-Open No. 2003-238414 discloses an antitumor agent containing, as an active ingredient, inositol phosphates (including inositol hexaphosphate). Besides, IP6 is known to have various activities for enhancement of immune system, prevention of kidney stone, reduction of cholesterol, and reduction of onset risk of coronary artery disease or diabetes. Vucenik I. et al., Nutrion and Cancer, 2006, 55, 109 describes that IP6 has a variety of actions such as antioxidation, enhancement of immune system, antiinflammation, modification of Phase I enzyme and Phase II enzyme, control of cancer gene, anti-angiogenesis effect, tumor metastasis inhibition, induction of apoptosis, enhancement of cell differentiation, and cell growth inhibition.

Although IP6 exhibits such many useful effects, it is difficult for IP6 to pass through a cell membrane because it has a large number of negative charges derived from six phosphate groups, and there is a limit of the amount of the cellular uptake of IP6. Besides, IP6 has the metal-chelating property, which results in a hindrance to mineral absorption in the body, and hence, it has been pointed that high intake thereof may cause an adverse reaction.

Japanese Translation of PCI International Application Publication No. 2009-541222 describes a method for preventing or treating acute short-term adverse health effects of ionizing radiation exposure in a mammal, comprising administering to the mammal an effective amount of a pharmaceutical composition comprising IP-6, its pharmaceutically acceptable salts, or its pharmaceutically acceptable derivatives, in any combination (claim 1). Paragraph 0015 of Japanese Translation of PCT International Application Publication No. 2009-541222 recites “as regards IP-6 and its derivatives, including pyrophosphate, and/or inositol, the subject matter of this application, those skilled in the art conclude: ‘Inositol hexaphosphate, IP-6, and its analog are entering testing as drugs. One of the challenges is to cover phosphates with protecting groups, to facilitate passage of the molecule into a cell. (DOE report of Jul. 13, 2005).’ Implied is the notion that IP-6 and its derivatives, including pyrophosphate, and/or inositol may currently be ineffective as radioprotectants.” Thus, it is mentioned that phosphate is covered with a protecting group for accelerating passage of a molecule of IP6 into a cell. The publication does not, however, suggest which protecting group is specifically used for covering phosphate, whether or not a compound having phosphate covered with a protecting group is really accelerated in the addition of a molecule to a cell, or what function the compound thus incorporated into the cell has.

A problem of IP6 is that IP6 is difficult to incorporate into a cell, and hence that there is a limit of the amount of the uptake thereof. However, a specific method for solving this problem has not been provided.

### CITATION LIST

### Patent Literature

PTL 1: Japanese Patent Laid-Open No. 2003-238414

PTL 2: Japanese Translation of PCT International Application Publication No. 2009-541222

### Non Patent Literature

NPL 1: Vucenik I, et al., Nutrion and Cancer, 2006, 55, 109

### SUMMARY OF INVENTION

### Technical Problem

An object of the present invention is to provide an IP6 derivative that is well incorporated into a cell and performs a function similar to that of IP6 in a living body. Another object of the present invention is to provide a pharmaceutical composition and a cosmetic composition containing the IP6 derivative.

### Solution to Problem

The present inventors have made earnest studies to solve the above-described problem, and as a result, have synthesized a phytic acid ester derivative, and thus, the present invention has been accomplished. The present invention includes, but not limited to, the following embodiments:

### EMBODIMENT 1

A compound represented by the following formula I:

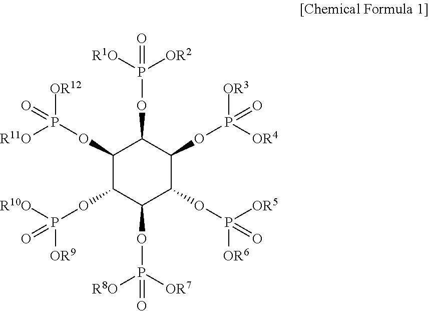

wherein R¹, R², R³, R⁴, R⁵, R⁶, R⁷, R⁸, R⁹, R¹⁰, R¹¹ and R¹² are each independently selected from the group consisting of H, the following formula II:

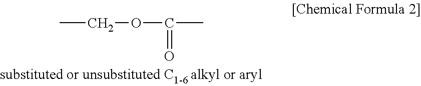

wherein —CH₂— is optionally substituted by one or two substituents, the following formula III:

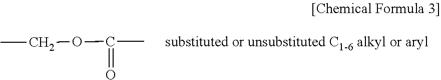

wherein —CH₂—C₆H₄— is optionally substituted by one or more of substituents, and the following formula IV:

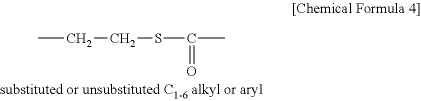

wherein —CH₂—CH₂— is optionally substituted by one or more of substituents, provided that a compound where all of R¹ to R¹² are H is excluded.

### EMBODIMENT 2

The compound according to embodiment 1, wherein the substituted or unsubstituted C₁₋₆ alkyl or aryl in formula II, formula III or formula IV is selected from the group consisting of substituted or unsubstituted methyl, substituted or unsubstituted ethyl, and substituted or unsubstituted butyl.

### EMBODIMENT 3

The compound according to embodiment 1 or 2, wherein six or more of R¹, R², R³, R⁴, R⁵, R⁶, R⁷, R⁸, R⁹, R¹⁰, R¹¹, and R¹² are not H.

### EMBODIMENT 4

The compound according to any one of embodiments 1 to 3, wherein none of R¹, R², R³, R⁴, R⁵, R⁶, R⁷, R⁸, R⁹, R¹⁰, R¹¹, and R¹² is H.

### EMBODIMENT 5

The compound according to any one of embodiments 1 to 4, wherein the C₁₋₆ alkyl or aryl is substituted by a substituent selected from the group consisting of halogen, C₁₋₄ alkyl, an amino group, a nitro group, a phenyl group, a hydroxyl group, a thiol group, C₁₋₄ acyl, and an allyl group.

### EMBODIMENT 6

The compound according to any one of embodiments 1 to 5, wherein one or more of —CH₂— in formula II, —CH₂—C₆H₄— in formula III, and —CH₂—CH₂— in formula IV are substituted by a substituent selected from the group consisting of halogen, C₁₋₄ alkyl, an amino group, a nitro group, a phenyl group, a hydroxyl group, a thiol group, C₁₋₄ acyl, and an allyl group.

### EMBODIMENT 7

The compound according to embodiment 1, wherein all of R¹, R², R³, R⁴, R⁵, R⁶, R⁷, R⁸, R⁹, R¹⁰, R¹¹, and R¹² are butyryloxymethyl or acetoxymethyl.

### EMBODIMENT 8

The compound according to any one of embodiments 1 to 7, wherein at least one of or all of R¹, R², R³, R⁴, R⁵, R⁶, R⁷, R⁸, R⁹, R¹⁰, R¹¹, and R¹² are charged to H through hydrolysis in a living body.

### EMBODIMENT 9

A pharmaceutical composition, comprising the compound of formula I according to any one of embodiments 1 to 8.

### EMBODIMENT 10

The pharmaceutical composition according to embodiment 9, having an activity selected from the group consisting of cell growth inhibition, cytotoxicity inhibition, enhancement of immune system, reduction of cholesterol, prevention of kidney stone, cancer metastasis inhibition, and fibrosis inhibition.

### EMBODIMENT 11

The pharmaceutical composition according to embodiment 9 or 10, wherein the pharmaceutical composition is for use in preventing or treating a disease selected from the group consisting of cancer, coronary artery disease, diabetes, and lithiasis.

### EMBODIMENT 12

The pharmaceutical composition according to embodiment 11, wherein the cancer is a cancer selected from the group consisting of leukemia, lymphoma, and myeloma.

### EMBODIMENT 13

The pharmaceutical composition according to any one of embodiments 9 to 12, wherein the pharmaceutical composition is orally administered, transdermally administered, intraperitoneally administered, or intravenously administered.

### EMBODIMENT 14

The pharmaceutical composition according to any one of embodiments 9 to 13, wherein the compound of formula I is hydrolyzed in a living body to change at least one of or all of R¹, R², R³, R⁴, R⁵, R⁶, R⁷, R⁸, R⁹, R¹⁰, R¹¹, and R¹² to H.

### EMBODIMENT 15

A use of the compound of formula I according to any one of embodiments 1 to 8 for production of a pharmaceutical composition.

### EMBODIMENT 16

A cosmetic composition comprising the compound of formula I according to any one of embodiments 1 to 8.

### EMBODIMENT 17

The cosmetic composition according to embodiment 16, having a skin-whitening effect or a skin-beautifying effect.

### EMBODIMENT 18

A laboratory reagent, containing the compound of formula I according to any one of embodiments 1 to 8.

### Advantageous Effects Of Invention

A phytic acid ester derivative (Pro-IP6) is more easily incorporated into a cell than IP6. Pro-IP6 incorporated into a cell exhibits, on a malignant cell such as a tumor cell, the same effects as those of IP6, such as the cytotoxic effect and an antitumor activity. Pro-IP6 is not toxic to a normal cell.

### BRIEF DESCRIPTION OF DRAWINGS

FIG. 1 illustrates results of measurement for methyl ester in a cell by LC/MS of IP6. The ordinate indicates relative intensity, and the abscissa indicates time (min).

FIG. 2 illustrates results of MTT assay using a MT-2 cell (HTLV transformed human T cell leukemia cell). The ordinate indicates the relative cell viability, wherein the cell viability obtained without adding IP6 or Pro-IP6 is regarded as 1. The abscissa indicates the concentration of IP6 or Pro-IP6 added.

FIG. 3 illustrates results of MTT assay using a M8166 cell (h T-lymphoblastoid cell). The ordinate indicates the relative cell viability, wherein the cell viability obtained without adding IP6 or Pro-IP6 is regarded as 1. The abscissa indicates the concentration of IP6 or Pro-IP6 added.

FIG. 4 illustrates results of MIT assay using a Jurkat cell (human T-lymphocyte cell). The ordinate indicates the relative cell viability, wherein the cell viability obtained without adding IP6 or Pro-IP6 is regarded as 1. The abscissa indicates the concentration of IP6 or Pro-IP6 added.

FIG. 5 illustrates results of MTT assay using a K562 cell (chronic myelogenous leukemia cell). The ordinate indicates the relative cell, wherein the viability obtained without adding IP6 or Pro-IP6 is regarded as 1. The abscissa indicates the concentration of IP6 or Pro-IP6 added.

FIG. 6 illustrates results of MTT assay using a PBMC (peripheral blood mononuclear cell) derived from a healthy person. The ordinate indicates the relative cell viability, wherein the cell viability obtained without adding Pro-IP6 is regarded as 1. The abscissa indicates the concentration of Pro-IP6 added.

FIG. 7 illustrates results of Western blotting on a Jurkat cell treated with Pro-IP6 or IP6 for 8 hours or 24 hours, using various antibodies, such as an anti-phospho-Akt (Thr 308) antibody, an anti-PARP-1 antibody, a Caspase-3 antibody, and an anti-β-actin antibody.

FIG. 8 illustrates the change of the tumor volume in an HTLV-1 infected cell-transplanted mouse after administration of Pro-IP6 or IP6, from day 1 to day 28 after transplantation. The tumor volumes on day 26 and day 2 of an IP administration group were extremely increased, which may be some kind of mistake, for example, some tumors might be measured together. A value on day 28 was obtained by measurement on the totally removed tumor, and hence is accurate.

FIG. 9 is a diagram illustrating a tumor volume in an HTLV-1 infected cell-transplanted mouse after administration of Pro-IP6 or IP6, on day 28 after transplantation.

FIG. 10 illustrates results of a cell viability test (flow cytometry) using a Jurkat cell, that is, a human T cell-derived leukemia cell.

FIG. 11 illustrates results of Western blotting on a Jurkat cell treated with Pro-IP6 or IP6 for 8 hours or 24 hours, using various antibodies, such as an anti-TRAF6 antibody (CST), an anti-pAMPK antibody (CST), and an anti-β-actin antibody (Sigma Aldrich).

FIG. 12 shows photographs of Hela cells with or without Pro-IP6 administered, the photographs obtained as 3D images under a fluorescence microscope, and indicates involvement of Pro-IP6 in aggregation of virus protein Gag or the MA region thereof.

### DESCRIPTION OF EMBODIMENTS

### 1. Phytic Acid Ester Derivative

The present invention relates to an ester derivative of a phytic acid (myo-inositol-1,2,3,4,5,6-hexaphosphate, IP6).

The ester derivative compound of phytic acid has a structure of the following formula I:

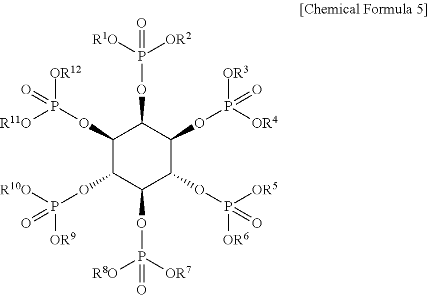

wherein R¹, R², R³, R⁴, R⁵, R⁶, R⁷, R⁸, R⁹, R¹⁰, R¹¹ and R¹² are each independently selected from the group consisting of H, the following formula II:

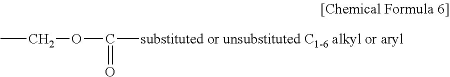

wherein —CH₂— is optionally substituted by one or two substituents, the following formula III:

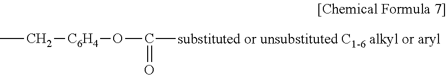

wherein —CH₂—C₆H₄— is optionally substituted by one or more substituents, and the following formula IV:

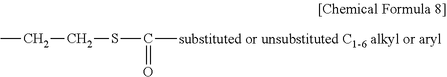

wherein —CH₂—CH₂— is optionally substituted by one or more substituents,

provided that a compound where all of R¹ to R¹² are H is excluded. If all of R¹ to R¹² are H, the compound corresponds to phytic acid (IP6).

In formula II, formula III or formula IV, alkyl of the substituted or unsubstituted C₁₋₆ alkyl may be linear or branched, and is preferably linear. Unlimited examples of the alkyl of the substituted or unsubstituted C₁₋₆ alkyl include methyl, ethyl, butyl, propyl, isopropyl, pentyl, t-butyl, and isobutyl. The substituted or unsubstituted C₁₋₆ alkyl may be selected from the group consisting of substituted or unsubstituted methyl, substituted or unsubstituted ethyl, and substituted or unsubstituted butyl, but not limited thereto.

The substituted or unsubstituted aryl includes an aromatic hydrocarbon group and polycyclic aromatic hydrocarbon group which are derived from a simple aromatic ring. The aryl is preferably an aromatic hydrocarbon group of a simple aromatic ring, namely, C₆ aryl, which has 6 carbon atoms. Unlimited examples of the substituted or unsubstituted aryl include a phenyl group, a benzyl group, a tolyl group, a xylyl group, and a naphthyl group.

If the C₁₋₆ alkyl or aryl is substituted, the number and the type of substituents are not especially limited. The C₁₋₆ alky or aryl may be substituted at all positions where it can be substituted, may be substituted at a single position, or may be unsubstituted. The C₁₋₆ alkyl or aryl may be substituted at a plurality of positions by the same substituents, or may be substituted by different substituents.

The type of a substituent is not especially limited. Unlimited examples include substituents such as halogen, C₁₋₄ alkyl, an amino group, a nitro group, a phenyl group, a hydroxyl group, a thiol group, C₁₋₄ acyl, and an allyl group. It may be a large substituent like a phenyl group. In one embodiment, the C₁₋₆ alkyl or aryl in the compound of formula I is substituted by a substituent selected from the group consisting of halogen, C₁₋₄ alkyl, an amino group, and a nitro group. Alkyl of the C₁₋₄ alkyl may be linear or branched, and is preferably linear. Unlimited examples include methyl, ethyl, butyl, propyl, and isopropyl.

Unlimited examples of halogen include fluorine, chlorine, bromine and iodine.

The C₁₋₄ alkyl may be linear or branched, and is preferably linear. Unlimited examples of the C₁₋₄ alkyl include substituted or unsubstituted methyl, ethyl, butyl, propyl, isopropyl, t-butyl, and isobutyl.

An amino group is a generic name of a monovalent substituent obtained by removing hydrogen from ammonia, primary amine, or secondary amine.

A thiol group is a substituent having hydrogenated sulfur at its end, and unlimited examples include a methanethiol group, an ethanethiol group, and a thiophenol group.

An acyl group is a substituent obtained by removing a hydroxyl group from oxoacid. Unlimited examples of the C₁₋₄ acyl include substituted or unsubstituted formyl, acetyl, propionyl, and butanoyl groups.

Each of —CH₂— in formula II, —CH₂—C₆H₄— in formula III, and —CH₂—CH₂— in formula IV may be substituted by one or more substituents if desired. In one embodiment, one or more of —CH₂— in formula II, —CH₂—C₆H₄— in formula III, and —CH₂—CH₂— in formula IV are substituted by one or more substituents. The number and the type of substituents are not especially limited. Each of —CH₂— in formula II, —CH₂—C₆H₄— in formula III, and —CH₂—CH₂— in formula IV may be substituted at all positions where it can be substituted, may be substituted at a single position, or may be unsubstituted. Each of —CH₂— in formula II, —CH₂—C₆H₄— in formula III, and —CH₂—CH₂— in formula IV may be substituted at a plurality of positions by the same substituents or by different substituents.

The type of substituents is not especially limited. Unlimited examples include substituents such as halogen, C₁₋₄ alkyl, an amino group, a nitro group, a phenyl group, a hydroxyl group, a thiol group, C₁₋₄ acyl, and an allyl group. It may be a large substituent like a phenyl group.

The definitions of halogen, C₁₋₄ alkyl, an amino group, a nitro group, a phenyl group, a hydroxyl group, a thiol group, C₁₋₄ acyl and an allyl group are the same as those described above.

In one embodiment, one or more of —CH₂— in formula II, —CH₂—C₆H₄— in formula III, and —CH₂—CH₂— in formula IV are substituted by a substituent selected from the group consisting of halogen, C₁₋₄ alkyl, an amino group and a nitro group. Alkyl of the C₁₋₄ alkyl may be linear or branched, and is preferably linear. Unlimited examples include methyl, ethyl, butyl, propyl, and isopropyl.

In the compound of formula I, the compound where all of R¹ to R¹² are H is not included. Preferably, two or more, three or more, four or more, five or more, six or more, seven or more, eight or more, nine or more, ten or more, or eleven or more of R¹, R², R³, R⁴, R⁵, R⁶, R⁷, R⁸, R⁹, R¹⁰, R¹¹, and R¹² are not H. The case of “not H” means that the compound is esterified by at least one group of R¹, R², R³, R⁴, R⁵, R⁶, R⁷, R⁸, R⁹, R¹⁰, R¹¹, and R¹². In one embodiment, six or more of R¹, R², R³, R⁴, R⁵, R⁶, R⁷, R⁸, R⁹, R¹⁰, R¹¹, and R¹² are not H.

Without limitation, in one embodiment, none of R¹, R², R³, R⁴, R⁵, R⁶, R⁷, R⁸, R⁹, R¹⁰, R¹¹, and R¹² is H. In one embodiment, all of R¹, R², R³, R⁴, R⁵, R⁶, R⁷, R⁸, R⁹, R¹⁰, R¹¹, and R¹² are butyryloxymethyl or acetoxymethyl.

As demonstrated in Example 2 described later, the compound of formula I (Pro-IP6) is well incorporated into a cell, and IP6 is then generated from Pro-IP6. Besides, Example 6 demonstrates that IP6 is generated from Pro-IP6 in a Jurkat cell to exhibit an antitumor activity similar to that of IP6. In one embodiment, the compound of formula I (Pro-IP6) is hydrolyzed in a living body, and at least one of or all of R¹, R², R³, R⁴, R⁵, R⁶, R⁷, R⁸, R⁹, R¹⁰, R¹¹, and R¹² are changed to H.

In one embodiment, the compound of formula I is myo-inositol hexaphosphate dodecakis(butyryloxymethyl) ester, or myo-inositol hexaphosphate dodecakis(acetoxymethyl) ester.

Herein, the ester derivative of phytic acid (myo-inostiol-1,2,3,4,5,6-hexaphosphate, IP6), namely, the compound of formula I, is generically designated as “Pro-IP6” in some cases, and also a specific compound represented by formula I is designated as “Pro-IP6” in some cases. The term “Pro-IP6” means a prodrug of IP6. Herein, the term “prodrug” refers to a compound that is metabolized, or converted into a biologically, pharmaceutically or therapeutically active form of the compound, after administration into a living body.

A synthesis method for the compound of formula I is not especially limited. A known method for esterifying a phosphate group can be employed. Those skilled in the art can appropriately employ a suitable method according to the types of R¹, R², R³, R⁴, R⁵, R⁶, R⁷, R⁸, R⁹, R⁹, R¹⁰, R¹¹, and R¹² in formula I. In one embodiment, for example, halogenated alkyl ester of carboxylic acid is synthesized from carboxylic acid. The resultant can be reacted with a basic salt of IP6 having a strongly basic substituent such as triethylamine and activated thereby, and thus, an ester derivative of IP6 can be obtained.

### 2. Pharmaceutical Composition

The present invention also relates to a pharmaceutical composition containing the compound of formula I.

The compound of formula I performs the function of an IP6 compound in a living body or in a cell. Without limitation, the pharmaceutical composition containing the compound of formula I has an activity selected from the group consisting of cell growth inhibition, cytotoxicity inhibition, enhancement of immune system, reduction of cholesterol, prevention of kidney stone, cancer metastasis inhibition, and fibrosis inhibition. These are all known as the activities of IP6.

Herein, the term “cell growth inhibition” means a function to stop growth of a cell. The actions of “cell growth inhibition” on a malignant cell including a cancer cell, and “cancer metastasis inhibition” can lead to a cytotoxic effect and an antitumor effect. Herein, Example 4 demonstrates that Pro-IP6 has an Akt phosphorylation inhibiting activity, and an apoptosis inducing activity. These activities have been reported also as a mechanism of the antitumor activity of IP6. Herein, the term “apoptosis inducing activity” refers to an activity capable of activating an apoptosis executing molecule, resulting in inducing apoptosis of a cell characterized by karyopyknosis, or the like.

Each of the terms “cell growth inhibition”, “cytotoxicity inhibition”, “prevention of kidney stone”, “cancer metastasis inhibition” and “fibrosis inhibition” means that the corresponding event can be inhibited as compared with a case where Pro-IP6 is not administered. Without limitation, it is inhibited preferably by 3% or more, 5% or more, 8% or more, 10% or more, 15% or more, 20% or more, or 25% or more.

The term “reduction of cholesterol” means that a ratio of incepting, into a living body, cholesterol contained in incepted food and drinks is reduced as compared with the case where Pro-IP6 is not administered. Without limitation, it is inhibited preferably by 3% or more, 5% or more, 8% or more, 10% or more, 15% or more, 20% or more, or 25% or more.

“Immunity” is one of homeostatic mechanisms of a living body of a human or an animal, the mechanisms including accumulation of a large number of mechanisms for protecting the living body from diseases by recognizing and killing a “foreign matter” (non-self substance) such as a pathogen, or an abnormal cell such as a cancer cell, in the living body. The term “enhancement of immune system” means that the ability of excluding a non-self substance or an abnormal cell is enhanced as compared with the case where Pro-IP6 is not administered. The immune system can be checked by measuring, for example, the number of leukocytes, the number of T cells (such as the number of CD4T cells, the number of CD8T cells, or the number of CD4/CD8T cells), the number of B cells, the number of NK cells and the like by a blood test, and comprehensively analyzing the results.

The pharmaceutical composition containing the compound of formula I is useful for preventing or treating a disease involving a condition selected from the group consisting of cell growth, cytotoxicity, weakened immune system, increase of cholesterol, kidney stone, cancer metastasis, and fibrosis. In one embodiment, the pharmaceutical composition containing the compound of formula I is a pharmaceutical composition for preventing or treating a disease selected from the group consisting of cancer, coronary artery disease, diabetes, and lithiasis.

The type of cancer is not especially limited. In one embodiment, cancer is cancer selected from the group consisting of leukemia, lymphoma, and myeloma. Other examples include a variety of cancers embracing liver cancer, glioma, neuroblastoma, sarcoma, and cancers of lung, colon, breast, bladder, ovarian, testis, prostate, testicular tumor, uterine, neck, pancreas, stomach, large intestine, small intestine and the other organs.

The “coronary artery disease” is a generic name of diseases caused when blood supply to myocardium is partially or completely blocked. The principal cause of the coronary artery disease is stenosis or occlusion caused in the coronary artery by arteriosclerosis due to cholesterol or the like accumulated on the inner wall of the coronary artery. Representative diseases included in the coronary artery disease include angina, and myocardial infarction (such as acute myocardial infarction).

“Diabetes” is a disease referring to a state where blood glucose level or hemoglobin A1c value exceeds a prescribed reference, it is classified into type 1 diabetes, type 2 diabetes, diabetes caused by a genetic abnormality such as maturity-onset diabetes of the young), secondary diabetes, gestational diabetes, and the like.

“Lithiasis” embraces kidney or urinary stones, stones in liver and bile transport route, salivary stone in a salivary gland, and the like. IP6 is known to be useful for treating and. preventing these stones.

The compound of formula I (Pro-IP6 compound) can be combined with a pharmaceutically or pharmacologically acceptable carrier if desired to be administered in the form of a pharmaceutical composition containing the compound of formula I. The number of types of pharmaceutically or pharmacologically acceptable carriers may be one or more. The number of types of the compound of formula I contained in the pharmaceutical composition may be one or more. A ratio of the compound of formula I contained in the pharmaceutical composition is not especially limited, and can be 0.01 to about 100% by mass, without limitation. The term “pharmaceutically” or “pharmacologically acceptable carrier” means an arbitrary carrier, diluent or excipient compatible with other components of a formulation and not harmful to a subject. Prescription of the pharmaceutical composition containing the compound of formula I based on the disclosure of the present application is encompassed in the technical field of the present invention.

The pharmaceutical composition containing the compound of formula I can be prescribed, for example, in accordance with the standard technique of the field of pharmaceuticals. See, for example, Alphonso Gennaro, ed., Remington's Pharmaceutical Sciences, 18th Ed., (1990) Mack Publishing. Co., Easton, Pa.

The route of administration of the pharmaceutical composition containing the compound of formula I is not especially limited. It may be prescribed in the form of a tablet, a capsule, a granule, a troche, a drink or the like to be orally administered. Alternatively, parenteral administration such as transdermal administration with a patch or the like, intraperitoneal administration, or intravenous administration with intravenous drip, injection, or the like may be employed. The administration can also be performed by intramuscular administration with intramuscular injection, enteral administration, topical administration or the like. In one embodiment, the pharmaceutical composition containing the compound of formula I is orally, transdermally, intraperitoneally, or intravenously administered. Specific examples of the prescription (formulation) of the pharmaceutical composition containing the compound of formula I are as follows.

For example, a tablet, a powder, a granule, a troche, a capsule or the like for oral administration can be produced by adding, to the pharmaceutical composition containing the compound of formula I, one or more solid inert ingredients such as an excipient, a disintegrating agent, a binder, and a lubricant, compression molding the resultant mixture, and subsequently coating the resultant, if necessary, for purposes of masking a taste, an enteric property, or persistence.

An injection can be produced, for example, by forming the pharmaceutical composition containing the compound of formula I together with, for example, a dispersant, a preservative, an isotonic agent or the like as an aqueous injection, or dissolving, suspending or emulsifying the pharmaceutical composition in a vegetable oil such as olive oil, sesame oil, cottonseed oil, or corn oil, propylene glycol or the like to form an oily injection.

An external preparation is produced, for example, by forming the pharmaceutical composition containing the compound of formula I into a solid, semi-solid or liquid composition. For example, the solid composition is produced by forming, into a powder, the pharmaceutical composition as it is, or as a mixture with an excipient, a thickener or the like. The liquid composition is produced, almost similarly to the injection, by obtaining an oily or aqueous suspension. A semi-solid composition may be in the form of an aqueous or oily gel, or an ointment. Besides, all of these compositions may contain a buffer, an antiseptic or the like. Examples of the external preparation include a cream, a lotion, a gel, and an ointment for topical administration.

A suppository is produced by, for example, forming the pharmaceutical composition containing the compound of formula I into an oily or aqueous solid, semi-solid or liquid. composition. Examples of an oily base to be used in such a composition include glyceride of higher fatty acids (such as cacao butter and Witepsols), intermediate fatty acids (such as Miglyols), and vegetable oils (such as sesame oil, soybean oil, and cottonseed oil). Examples of an aqueous gel base include natural gums, a cellulose derivative, a vinyl polymer, and an acrylic acid polymer.

The pharmaceutical composition containing the compound of formula I may further contain one or more other active ingredients if desired. “Other active ingredients” may be a component having an activity selected from the group consisting of cell growth inhibition, cytotoxicity inhibition, enhancement of immune system, reduction of cholesterol, prevention of kidney stone, cancer metastasis inhibition, and fibrosis inhibition similarly to the compound of formula I. Alternatively, a component having an activity different from these may be used.

To the pharmaceutical composition containing the compound of formula I, a stabilizer, an antioxidant, and a preservative may be further added if desired. Examples of an appropriate antioxidant include sulfurous acid, ascorbic acid, citric acid and a salt thereof, and sodium EDTA. Examples of an appropriate preservative include benzalkonium chloride, methyl- or propyl-paraben, and chlorobutanol.

A dose and an administration period of the pharmaceutical composition containing the compound of formula I are determined depending on specific situations of an individual patient, such as the size, the mass, the age and the sex of the administration target, characteristics and the stage of a disease to be treated, aggression of the disease, the administration route, and specific toxicity of radiation. The dose and the administration period can be determined experimentally using a known test protocol, or by an extrapolation method based on in vivo or in vitro test data. The concentration ranges described herein are for merely illustrative purpose only, and does not restrict the scope or practice of the claimed composition.

For example, the dose of the pharmaceutical composition containing the compound of formula I is, in terms of the amount of the compound of formula I as the active ingredient, about 0.01 to about 2000 mg/kg/day, and more preferably about 0.05 to about 1000 mg/kg/day. A dose of about 1.0 to about 200 mg/kg/day, for example, about mg/kg/day is a particularly suitable dose. The pharmaceutical composition may be administered once a day, or may be divided into some low doses to be administered simultaneously or at time interval. The dose may be dividedly administered in a plurality of times, for example, administered twice each at a divided dose of 25 mg/kg. Alternatively, a higher or lower dose may be employed.

In one embodiment, the compound of formula I (Pro-IP6) contained in the pharmaceutical composition is hydrolyzed in a living body, and at least one of or all of R¹, R², R³, R⁴, R⁵, R⁶, R⁷, R⁸, R⁹, R¹⁰, R¹¹, and R¹² are changed to H.

The present invention also relates to a use of the compound of formula I for production of a pharmaceutical composition.

The present invention further relates to a method for imparting, to a target, an activity selected from the group consisting of cell growth inhibition, cytotoxicity inhibition, enhancement of immune system, reduction of cholesterol, prevention of kidney stone, cancer metastasis inhibition, and fibrosis inhibition, the method including administering the compound of formula. Ito the target in need thereof.

The present invention further relates to a method for preventing or treating a disease involving a condition selected from the group consisting of cell growth, cytotoxicity, weakened immune system, increase of cholesterol, kidney stone, cancer metastasis, and fibrosis, the method including administering the compound of formula I to a target in need thereof. Examples of the “disease involving a condition selected from the group consisting of cell growth, cytotoxicity, weakened immune system, increase of cholesterol, kidney stone, cancer metastasis, and fibrosis” include cancer, coronary artery disease, diabetes, and lithiasis.

The present invention also relates to a method for preventing or treating a disease selected from the group consisting of cancer, coronary artery disease, diabetes, and lithiasis, the method including administering the compound of formula Ito a target in need thereof.

In Example 3, it was revealed that Pro-IP6 is not toxic to a normal cell. A composition containing a compound of formula I can be safely used in a living body, and is useful for a pharmaceutical composition or a cosmetic composition.

### 3. Cosmetic Composition

The present invention relates to a cosmetic composition containing the compound of formula I. In one embodiment, the cosmetic composition has a skin-whitening effect or a skin-beautifying effect.

The form and the amount of the cosmetic composition used are not especially limited. It can be in the form of a basic skin care item such as a toner (lotion), an emulsion (milky lotion), a cream, an essence, a gel, a pack, or a mousse.

The formulation of the cosmetic composition is not especially limited, and it may contain any component as long as it is acceptable as a cosmetic. In the cosmetic composition, water, polyhydric alcohol, a water-soluble polymer compound, an oil-soluble component (oil or wax), an antiseptic, an antioxidant, a perfume and the like which are used in a cosmetic composition can be blended if necessary.

The cosmetic composition may contain polyhydric alcohol for performing a moisturizing function, a viscosity-adjusting function or the like. Besides, addition of polyhydric alcohol can lower the water activity of the cosmetic composition to suppress microbial growth.

In the cosmetic composition, a water-soluble polymer compound may be blended. As the water-soluble polymer compound that can be blended, any of synthetic polymers, natural polymers, and semi-synthetic polymers can be widely used. In particular, sugars, proteins, and complexes of these are preferred.

The cosmetic composition may contain an oil-soluble component dissolved in an oil median. As the oil-soluble component, other components usually used as a UV absorber, an antioxidant, an anti-inflammatory agent, a moisturizer, a hair protectant, a dispersant, a solvent, a skin whitening agent, an anti-spot agent, a cell activator, an emollient agent, a keratolytic agent, an antistatic agent, vitamins, a metabolic syndrome improving agent, a hypotensive agent, a sedative and the like can be also used. Examples include fats and oils such as olive oil, camellia oil, macadamia nut oil, and castor oil; hydrocarbons such as liquid paraffin, paraffin, Vaseline, ceresin, microcrystalline wax, and squalane; waxes such as carnauba wax, candelilla wax, jojoba oil, beeswax, and lanolin; esters such as isopropyl myristate, 2-octyldodecyl myristate, cetyl 2-ethylhexanoate, and diisostearyl malate; fatty acids such as palmitic acid stearic acid, and isostearic acid; higher alcohols such as cetyl alcohol, stearyl alcohol, isostearyl alcohol, and 2-octyldodecanol; silicone oils such as methyl polysiloxane, and methyl phenyl polysiloxane; fatty acid esters of glycerin; and other polymers, oil-soluble pigments, and oil-soluble proteins. In addition, the examples include various vegetable-derived oils and animal-derived oils that are mixtures of these.

In the cosmetic composition, an antioxidant may be blended for retaining stability, A usable antioxidant is not especially limited, and examples include a group of compounds consisting of polyphenols, and radical scavengers.

The cosmetic composition may contain a perfume. As the perfume, any one of natural perfumes of animal, plant and mineral bases, and synthetic perfumes can be used.

The present invention also relates to a use of the compound of formula I for production of a cosmetic composition.

### 4. Composition

The present invention also relates to a composition containing the compound of formula I.

In one embodiment, the composition containing the compound of formula I can be used as a laboratory reagent. In one embodiment, the composition can be used as a laboratory reagent for elucidating intracellular signal transduction of IP6.

Since IP6 is little incorporated singly into a cell, it is necessary to use a large amount thereof for a use as a reagent. IP6 has, however, a negative charge, and is an acidic substance, and therefore, there is a possibility that an experimental environment and a target cell may be affected. When the laboratory reagent of the present invention is used, even a small amount of the reagent can be introduced into a cell, and in addition, owing to protection by a phosphate group, the influence on the experimental environment can be minimized. In other words, when the laboratory reagent of the present invention is added to a cell, IP6 can be efficiently introduced into the cell.

### EXAMPLES

Now, the present invention will be described in detail based on examples, and it is noted that the present invention is not limited to these examples. Those skilled in the art can easily change/modify the present invention on the basis of the present description, and such change and modification are embraced in the technical scope of the present invention.

### Example 1

### Synthesis of Myo-Inositol-Hexaphosphate Dodecakis(Butyryloxymethyl) Ester

In this example, myo-inositol hexaphosphate dodecakis(butyryloxymethyl) ester was synthesized in accordance with the following route. First, butyric acid was used as a raw material of the synthesis to synthesize bromomethyl butyrate in two stages. The resultant was reacted with IP6 triethylamine salt to obtain butyryloxymethyl ester of IP6 (Pro-IP6). Ultimately, Pro-IP6 was purified by HPLC.

### Chemical Formula 9

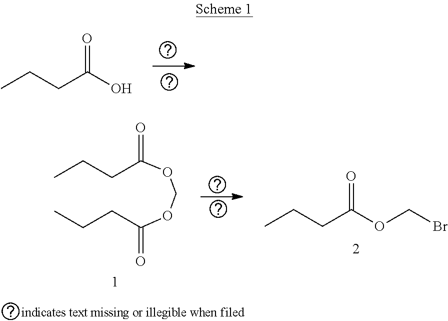

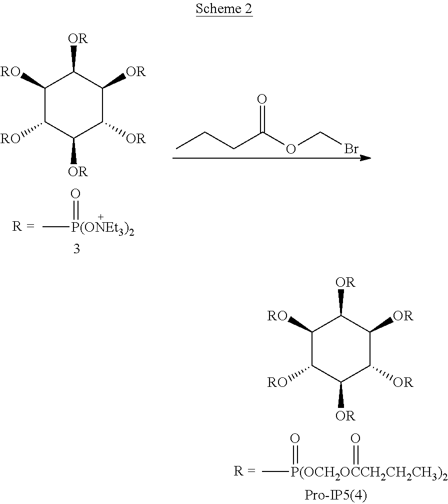

### Synthesis of Methylene Dibutyrate (1)

A 2M NaOH aqueous solution (30 ml) was added to butyric acid (2.65 g=2.75 30.1 mmol), followed by stirring for 30 minutes. Thereafter, tetrabutylammonium hydrogen sulfate (10.2 g, 30.5 mmol) was added thereto, the resultant was stirred for 30 minutes, and an aqueous layer was extracted with CH₂Cl₂ (50 ml×4). An organic layer was dried over magnesium sulfate, and the resultant was filtered and refluxed at 45° C. for 2 days. The resultant was concentrated by evaporating CH₂Cl₂, and then dissolved in hexane (60 ml), separated with 10% acetic acid (50 ml), water (50 ml×2) and a saturated saline solution (50 ml), and dried over magnesium sulfate. After the drying, a filtrate was concentrated with an evaporator to obtain a colorless liquid compound, methylene dibutyrate (1) (1.354 g, 96%).

¹H NMR (600 MHz, CDCl₃) δ: 0.96 (t, 6H, CH₃), δ: 1.66 (sext, 4H, CH₂CH₃), δ: 2.34 (t, 4H, CH₂CH₂CH₃), δ: 5.76 (s, 2H, OCH₂O)

### Synthesis of Butyric Acid Bromomethyl Ester (2)

To the compound (1) (0.47 g. 2.52 mmol), bromotrimethylsilane (TMSBr) (0.77 g=0.65 ml, 5.03 mmol) and zinc bromide (29.6 mg, 0.13 mmol) were added, followed by stirring for 1 day under argon atmosphere. Thereafter, the resultant reaction solution was dissolved in diethyl ether (20 ml), 1 M hydrochloric acid (10 ml) was added thereto, and the resultant was stirred for 10 minutes. An aqueous layer was removed, and a saturated sodium carbonate solution (20 ml) was added thereto, followed by stirring for 30 minutes. Thereafter, an aqueous layer was removed, and an organic layer was washed with water (50 ml×2) and a saturated saline solution (50 ml), and the resultant was dried over magnesium sulfate. After the drying, Kugelrohr distillation was performed to obtain a first fraction (1 mmHg, 30° C.) of first 5 minutes, and then, a major fraction (1 mmHg, 55° C.) flowing for 15 minutes was collected to obtain a colorless liquid, butyric acid bromomethyl ester (2) (21.4 g, 47%). It is noted that the compound (2) is easily decomposed, and hence was immediately used in the following reaction.

¹H NMR (600 MHz, CDCl₃) δ: 0.96 (t, 3H, CH₃), δ: 1.67 (sext, 2H, CH₂CH₃), δ: 2.35 (t, 2H, CH₂CH₂CH₃), δ: 5.80 (s, 2H, OCH₂Br)

### Synthesis of Myo-Inositol Hexaphosphate Et₃N Salt (3)

Myo-inositol hexaphosphate Na salt (available from Sigma Aldrich) (1.00 g, 1.51 mmol) was purified with a cation exchange resin (manufactured by Wako Pure Chemical Industries Ltd., Dowex 50WX8 (100-200 mesh), and Et₃N (3 ml) was added to the resultant to obtain a colorless solid, myo-inositol hexaphosphate Et₃N salt (3) (2.6 g, 92%).

### Synthesis of Myo-Inositol Hexaphosphate Dodecakis(Butyryloxymethyl Ester (4)

The compound (3) (50.0 mg, 2.69×10⁻² mmol) was dissolved in acetonitrile (5 ml) by azeotropy with acetonitrile (5 ml×3 times), and DIPEA (0.2 ml) was added to the resultant, followed by stirring for 16 hours under argon atmosphere. Thereafter, the resultant was dissolved in acetonitrile (5 ml) by azeotropy with acetonitrile (5 ml×3 times), and the compound (2) (0.24 g, 1.35 mmol) and DIPEA (0.2 ml) were added thereto, followed by stirring for 3 days under argon atmosphere. After the stirring for 3 days, the resultant was purified by HPLC (MeOH: H₂O=96:4, F=3 ml/min, rt=6.3 min) to obtain a colorless liquid, myo-inositol hexaphosphate dodecakis(butyryloxymethyl) ester (4) (8.2 mg, 16%). Hereinafter, myo-inositol hexaphosphate dodecakis(butyryloxymethyl) ester (4) was used as an example of “Pro-IP6” in the following examples.

¹H NMR (600 MHz, CD₃CN) δ: 0.98 (m, 36H, CH₃), δ: 1.66 (m, 24H, CH₂CH₃), δ: 2.41 (m, 24H, CH₂CH₂CH₃), δ: 4.55 (q, 1H), δ: 4.63 (d, 2H), δ: 4.75 (q, 2H), δ: 5.29 (d, 1H), δ: 5.68 (m, 24H, OCH₂O).

¹³C NMR (600 MHz, CD₃CN) δ: 12.5, 17.4, 34.9, 72.4, 74.5, 75.3, 82.8, 171.6.

HRMS (FAB+) m/s calcd (C₆₆H₁₁₄O₄₈P₆): 1860.4905 found 1883.4766

### Example 2

### Quantitative Determination of IP6 in Cell

In this example, incorporation of Pro-IP6 into a HeLa cell was quantitatively determined.

HeLa cells (5×10⁵ cells/well culture fluid: 1 mL) were seeded in a 3.5 cm dish to be cultured for 24 hours. Thereafter, DMSO (1%, Fujifilm Wako Pure Chemical Corporation), an aqueous solution (1%) of IP6 (final concentration: 10 μM, Sigma Aldrich), or a DMSO solution (1%) of Pro-IP6 (final concentration: 10 μM) synthesized in Example 1 was added thereto, and the resultant was cultured for another 30 minutes. HeLa cells were cultured with neither IP6 nor Pro-IP6 added, followed by adding the same amount of DMSO thereto, and the resultant was used as a negative control.

Thereafter, the resultant was washed with 1×PBS (−) once, and the resultant cells were scraped with a cell scraper, transferred to a 1.5 mL tube, and washed with 1×PBS (−) three times. To the thus obtained precipitate, 350 μl of MeOH (Fujifilm Wako Pure Chemical Corporation) and 1% NP-40 (Nacalai Tesque, Inc.) were added, followed by sonication (10 minutes). After equilibrating a column (Oasis WAX 1 cc, Waters) with MeOH (1 mL) and H₂O (1 mL), a disrupted cell extract was added thereto. Thereafter, the column was washed with 50% MeOH (1 mL), and eluted with 50% MeOH (200 μL×2) containing 2 mol/L of HCl (Fujifilm Wako Pure Chemical Corporation). The thus obtained eluate was concentrated, and dissolved in MeOH (50 μL), and trimethylsilyldiazomethane (200 μl, Tokyo Chemical industry Co., Ltd.) was added thereto, followed by stirring at 50° C. for 1 hour under argon atmosphere. The reaction was stopped by adding H₂O (100 μL). Through a reaction of trimethylsilyidiazomethane with IP6, IP6 methyl ester is obtained.

The thus obtained sample (IP6 methyl ester) was measured by LC/MS [column: MastroC18 2.1 mm×100 mm, 3 m (Shimadzu GLC Ltd.), mobile phase: 0.1% formic acid solution (Fujifilm Wako Pure Chemical Corporation), acetonitrile (Fujifilm Wako Pure Chemical Corporation)] [LC:LC-20AD (Shimadzu), MS: amaZon speed (BRUKER)].

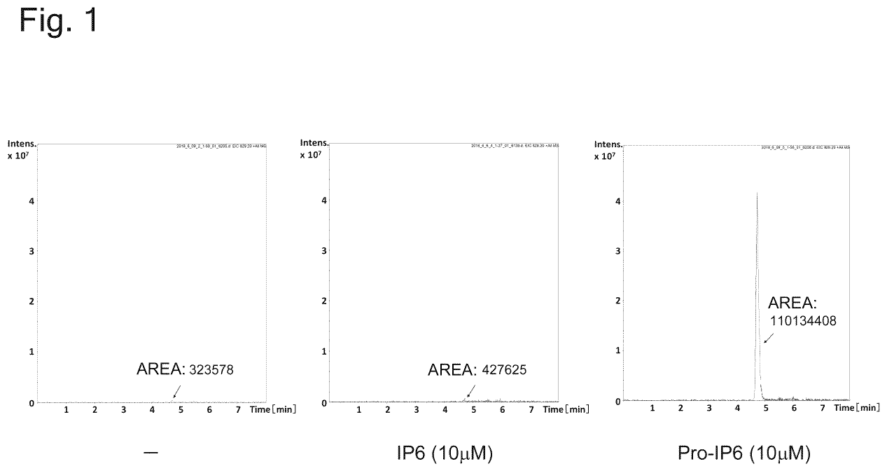

Representative data is illustrated in FIG. 1. As illustrated in FIG. 1, the value of IP6 was substantially the same as that of the negative control, and thus, IP6 was not substantially incorporated into the Hela cells. On the contrary, in using Pro-IP6, IP6 methyl ester in an amount not less than 200 times of that of IP6 was observed. Thus, it was revealed that Pro-IP6 is well incorporated into a Hela cell, and that IP6 is generated from Pro-IP6 after being incorporated into the cell.

### Example 3

### MTT Assay

In this example, the cytotoxic effect of Pro-IP6 on four types of blood cancer cell lines and a cell derived from a healthy person was examined by MTT assay. MTT assay is a colorimetric determination method for measuring enzyme activity for reducing MTT (3-(4,5-di-methylthiazol-2-yl)-2,5-diphenyltetrazolium bromide) into a formazan pigment (violet). It is possible to check viability of cultured cells by MTT assay.

Cancer cell lines (1.74×10⁴ cells/well, culture fluid: 200 μL)
MT-2 cell (HTLV transformed human T cell leukemia cell)
M8166 cell (human T-lymphoblastoid cell)
Jurkat cell (human T-lymphocyte cell)
K562 cell (chronic myelogenous leukemia cell)

Cell derived from healthy person (5.0×10⁵ cells/well, culture fluid: 200 μL)

### PBMC (Peripheral Blood Mononuclear Cell)

Each of the above-described types of cells was seeded in a 96-well plate, and was cultured for 8 hours for each of the various cancer cell lines and for 16 hours for PMBC. Thereafter, DMSO (1%, Fujifilm Wako Pure Chemical Corporation), an aqueous solution (1%) of IP6 (final concentration: 1 μM or 10 μM, Sigma Aldrich), or a DMSO solution (1%) of Pro-IP6 (final concentration: 1 μM or 10 μM) was added thereto, followed by culturing for 8 hours or 24 hours. An MTT reagent (Dojindo Laboratories, 1.1 mg/mL) was added in an amount of 50 μL per well, and the resultant was cultured for another 4 hours. The thus precipitated crystal was collected, a supernatant was removed, and 100 μL of DMSO was added thereto to completely dissolve. The resultant solution was measured for an absorbance (550 nm).

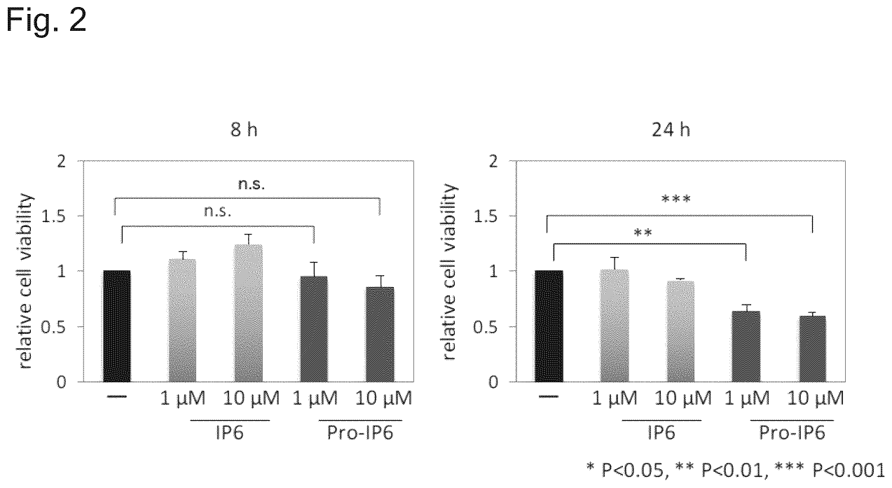

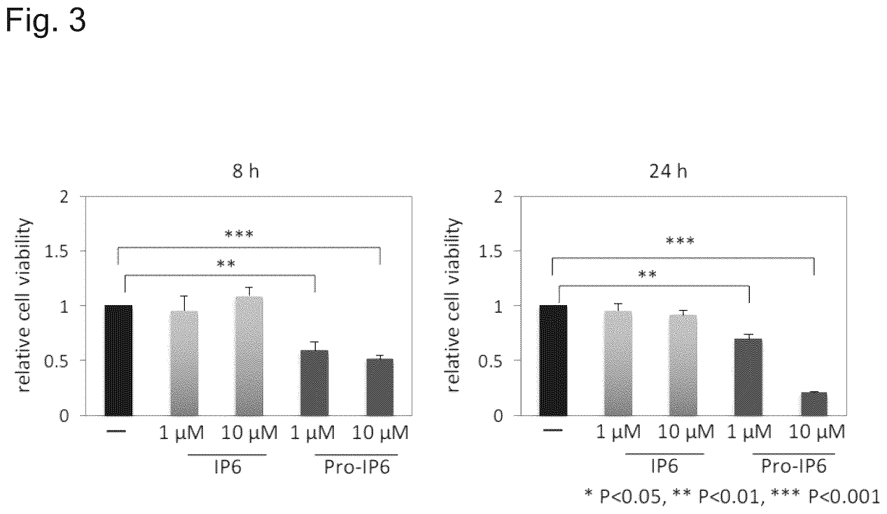

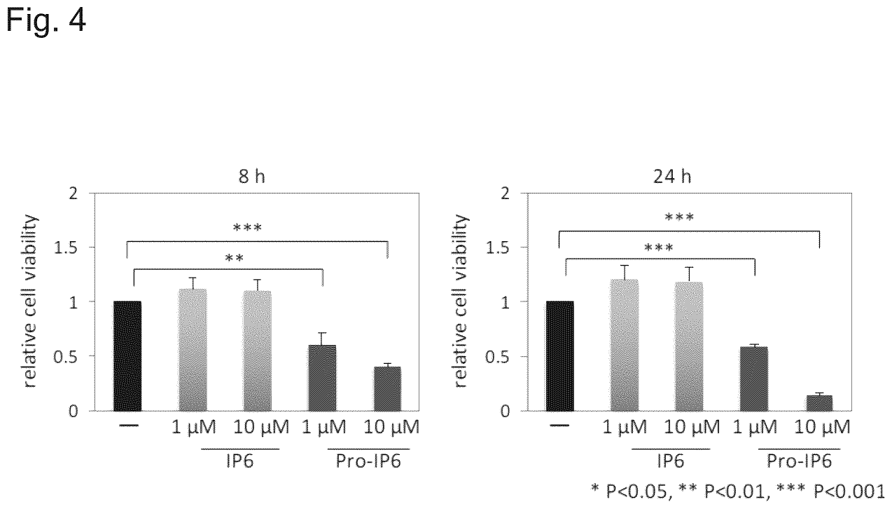

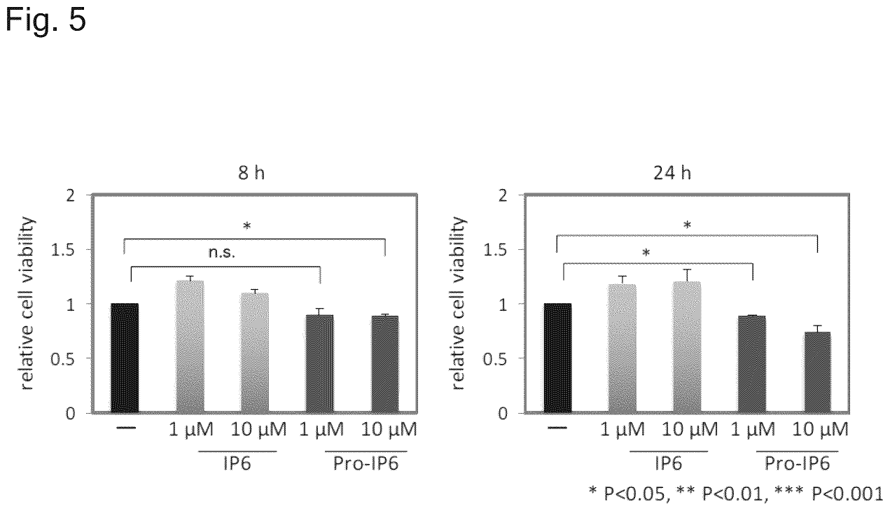

Results are illustrated in FIG. 2 to FIG. 6. As is understood from FIG. 2 to FIG. 5, in this example, the administration of Pri-IP6 lowered the relative cell viability of the blood cancer cell lines by 25% or more, and thus, Pro-IP6 exhibited the cytotoxic effect on any of the blood cancer cell lines. On the contrary, IP6 minimally exhibited the effect. Among the cancer cell lines tested, the effect was particularly strong on M8166 and Jurkat, and most of the cells were killed at 10 μM.

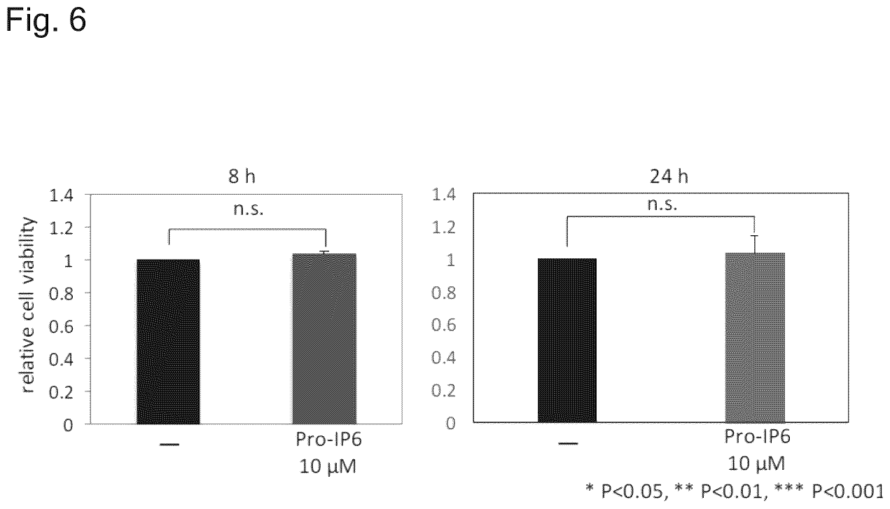

Pro-IP6 thus exhibited the cytotoxic effect on the cancer cells, but was not toxic to the peripheral blood mononuclear cell (PBMC) of a healthy person even at 10 μM (FIG. 6).

### Example 4

### Western Blotting (1)

In this example, the action mechanism for killing cells by Pro-IP6 was studied by Wester blotting using a Jurkat cell and various antibodies.

Jurkat cells (1×10⁵ cells/well, culture fluid: 500 μL) were seeded in a 24-well plate to be cultured overnight. Thereafter, DMSO (1%, Fujifilm Wako Pure Chemical Corporation), an aqueous solution (1%) of IP6 (final concentration: 10 μM, Sigma Aldrich), or a DMSO solution (1%) of Pro-IP6 (final concentration: 1 μM or 10 μM) was added thereto, followed by culturing for 8 hours or 24 hours. A supernatant was removed, the resultant was washed with 1×PBS (−) once, Laemmli sample buffer was added thereto, and the resultant was boiled at 100° C. for 1 hour to dissolve the cells. The resultant was subjected to electrophoresis and transferred onto a membrane (Millipore), and was reacted with, as a primary antibody, an anti-phospho-Akt (Thr308) antibody (CST), an anti-PARP-1 antibody (Merck), a Caspase-3 antibody (CST), or an anti-β-actin antibody (Sigma Aldrich). Detection was performed using ImmunoStar LD (Fujifilm Wako Pure Chemical Corporation).

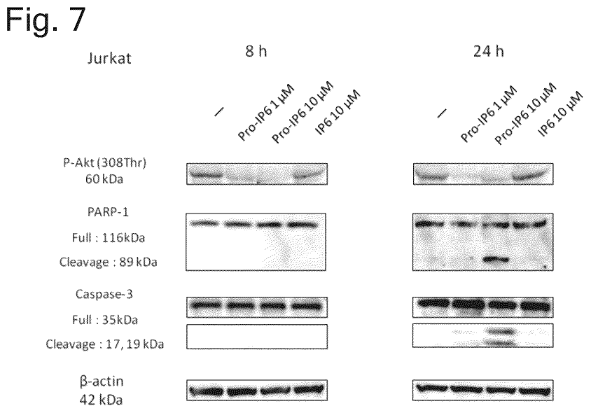

Results are illustrated in FIG. 7, It was found that Pro-IP6 at 1 μM and 10 μM inhibits phosphorylation of Akt (FIG. 7). Besides, at a concentration of 10 μM, cleavage of PARP-1 and Caspase-3 was observed, which indicates induction of apoptosis. The inhibition of phosphorylation of Akt and the induction of apoptosis have been reported as the mechanism of the antitumor activity of IP6. Accordingly, the results obtained in this example suggest that IP6 is generated from Pro-IP6 in a Jurkat cell to exhibit the antitumor activity similar to that of IP6.

### Example 5

### Effect on HTLV-1 Infected Cell-Transplanted Mouse

In this example, the in vivo effect of Pro-IP6 on an HTLV-1 infected cell-transplanted mouse was checked. HTLV-1 (human T cell leukemia virus 1) mainly infects CD4-positive T lymphocyte, and when the infected cell cancerates, refractory malignant tumor, adult T-cell leukemia (ATL) is caused. In this example, S1T cell, that is, one of ATL cell lines, was used.

### (5-1) Mouse

5-Week-old female NSG mice (NOD, Cg-Prkdc scid I12rg tm1Wj1/SzJ, Charles River Laboratories Japan, Inc.) were used. Five mice each in each of two cages, namely, ten mice in total, were raised with sterilized bedding (Pepar Clean: Japan SLC, Inc.) placed in a sterilized cage (Clea Japan, Inc.). The mice were fed with γ-ray irradiated diet, and were allowed to free access to hydrochloric acid water prepared by adding hydrochloric acid to autoclaved sterile water with retaining pH 2.5 to 3.0. The mice were habituated for 1 week upon arrival. This study was made in accordance with Safety Management Standards for Gene Recombinant Experiments and Rakuno Gakuen University Guidelines on Animal Experiments under approval of Safety Committee for Gene Recombinant Experiments (Approval No. 154) and Animal Experiment Committee (Approval No. VH16A21).

### (5-2) Administered Agents

The following agents were used:

1) DMSO, as negative control

2) IP6 (71 mg/ml in H₂O), MW: 660, comparative substance

3) pro-IP6 (200 mg/ml in DMSO), MW: 1861, synthesized compound

The agent to be administered to each mouse was prepared as follows. A dose per day was set to 20 mg/kg.

1. 2 μl of DMSO+198 μl of saline

2. 2 μl of IP6+196 μl of saline+2 μl of DMSO (final concentration: 7.1 mg/kg: 11 μmol/kg, DMSO 1%)

3. 2 μl of pro-IP6+198 μl of saline (final concentration: 20 mg/kg: 11 μmol/kg, DMSO 1%) 0.2 ml (DMSO 1%)

### (5-3) Cell For Transplantation

S1T cell, that is, one of ATL cell lines, was used. On the day of transplantation, about 2.5 L of S1T cell culture fluid (day 4 after passage) was prepared, and was dispensed to six 500 ml centrifuge tubes. The culture fluid was centrifuged with HITACHI-himac-SCR20B at 1500 rpm for 5 minutes at 4° C. A supernatant was discarded, the resultant was floated in 120 ml of Dulbecco PBS (D-PBS: Wako Pure Chemical Industries Ltd.), and the resultant cells were transferred to four 50 ml centrifuge tubes, and centrifuged with TOMY-RL-101 at 1000 rpm for 5 minutes at 4° C. A supernatant was discarded, the resultant was washed with 120 ml of D-PBS again, and floated in 5 ml of D-PBS to count the number of cells, and the resultant was adjusted using D-PBS to attain 0.2 ml: 5×10⁷/mouse.

### (5-4) Transplantation Experimental Protocol

The S1T cell line (0.2 ml: 5×10⁷/cells/mouse) was subcutaneously transplanted in the neck of each of the ten 6-week-old NSG mice having been habituated for 1 week to obtain an ATL model mouse. The thus obtained ten ATL model mice were randomly divided into three groups. The groups were a control group consisting of three mice, an IP6 administration group consisting of three mice, and a pro-IP6 administration group consisting of four mice.

The control group was dosed with DMSO, and the IP6 administration group and the pro-IP6 administration group were intraperitoneally dosed respectively with IP6 in an amount of 7.1 mg/kg and pro-IP6 in an amount of 20 mg/kg. The mice were dosed continuously for 28 days after the day of the cell transplantation, and the weight and the tumor volume of each mouse were measured every day. The tumor volume was defined as (shorter diameter in mm)²×(longer diameter in mm)×0.5, and the shorter diameter and the longer diameter were measured with a caliper. On day 28 after the cell transplantation, all the mice were euthanized under excessive isoflurane anesthesia. Subsequently, the tumor was totally removed, and the tumor volume and the tumor weight of each tumor were measured.

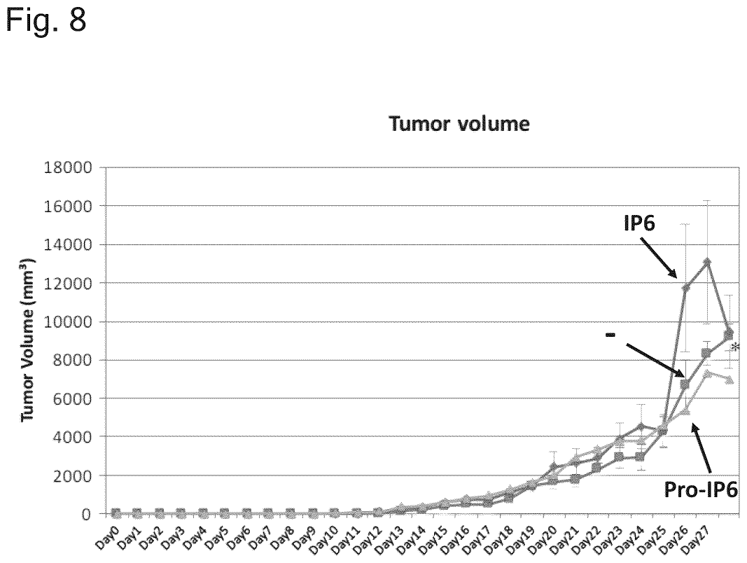

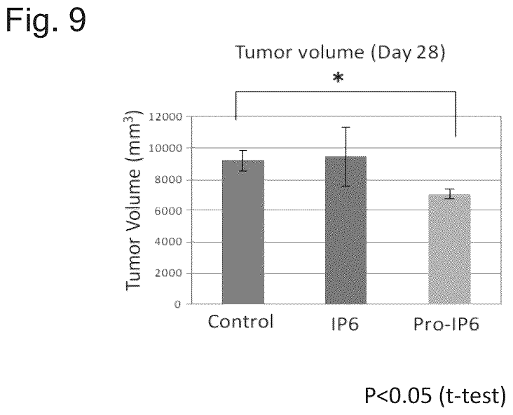

A result of the thus obtained change in the tumor volume from day 1 to day 28 after the transplantation is illustrated in FIG. 8. The tumor volumes on day 28 after the transplantation are illustrated in FIG. 9. As illustrated in FIG. 9, the tumor volume of the Pro-IP6 administration group is smaller by about 25% as compared with that of the control group on day 28 after transplantation, and it was revealed that Pro-IP6 has the antitumor effect. It is noted that the tumor volume of the IP administration group was not significantly different from that of the control group.

### Example 6

### Synthesis of Myo-Inositol Hexaphosphate Dodecakis(Acetoxymethyl) Ester

From myo-inositol hexaphosphate Et₃N salt (3) synthesized in Example 1, myo-inositol hexaphosphate dodecakis(acetoxymethyl) ester was synthesized as follows.

Specifically, the compound (3) (176.8 mg, 9.44×10⁻² mmol) was dissolved in acetonitrile (2 ml) by azeotropy with acetonitrile (5 ml×3 times), and DIPEA (0.2 ml) was added thereto, and the resultant was concentrated by stirring for 30 minutes under argon atmosphere. Thereafter, the resultant was dissolved in acetonitrile (3.0 ml), and bromomethyl acetate (1.0 g, 18.9 mmol) and DIPEA (0.4 ml) were added thereto, followed by stirring for 3 days under argon atmosphere. After the stirring for 3 days, the resultant was purified by HPLC (ACN: H₂O=30:70 (0 min to 10 min) ACN: H₂O=95:5 (10.1 min to min), F=3 ml/min, rt=15.0 min) to obtain a colorless liquid, myo-inositol hexaphosphate dodecakis(acetoxymethyl) ester.

¹H NMR (600 MHz, CD₃CN) δ: 2.14 (m, 36H, OCOCH₃), δ: 4.55 (q, 1H), δ: 4.63 (t, 2H), δ: 4.75 (q, 2H), δ: 5.27 (d, 1H), δ: 5.68 (m, 24H, OCH₂O).

HRMS (FAB+) m/s calcd (C₄₄H₆₆O₄₈P₆Na): 1547.1047. found 1547.1041

### Example 7

### Cell Viability Test (Flowcytometry)

In this example, a cell viability test (flowcytometry) was performed using a Jurkat cell, that is, a human T cell-derived leukemia cell.

Jurkat cells (1×10⁵ cells/well, culture fluid: 500 μL) were seeded in a 24-well plate (Iwaki) to be cultured overnight. Thereafter, DMSO (1%, Fujifilm Wako Pure Chemical Corporation), an aqueous solution (1%) of IP6 (final concentration: 10 μM, Sigma Aldrich), or a DMSO solution (1%) of Pro-IP6 (myo-inositol hexaphosphate dodecakis(butyryloxymethyl) ester (4) synthesized in Example 1) (final concentration: 10 μM) was added thereto, and the resultant was cultured for 24 hours. A supernatant was removed, the resultant was washed with 1×PBS (−) once, 100 μL of 5× binding solution (PromoCell) was added thereto, and Annexin V-FITC (PromoCell) and 7-AAD (Becton, Dickinson and Company) were added thereto to perform a reaction for 15 minutes. Thereafter, measurement was performed using FACS Calibur (Becton, Dickinson and Company), and analysis was performed using FlowJo (Becton, Dickinson and Company).

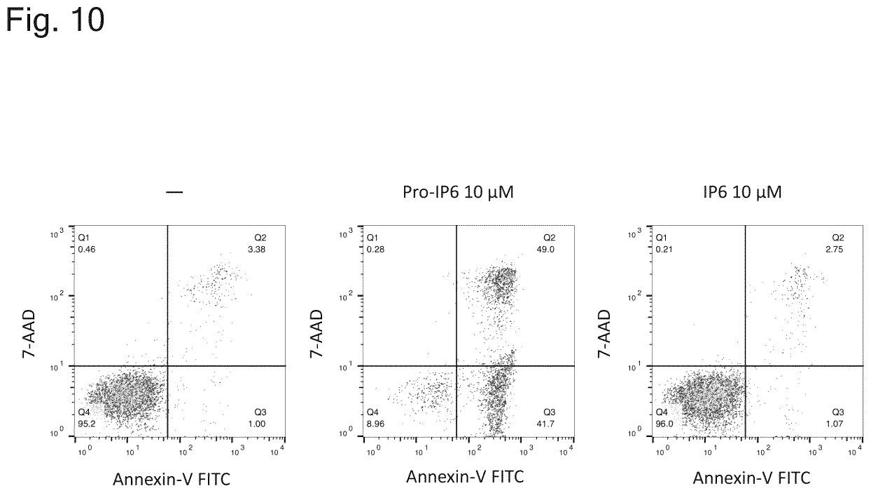

Results are illustrated in FIG. 10. When Pro-IP6 was added, the induction of apoptosis in a Jurkat cell, that is, human T cell-derived leukemia cell, was observed.

### Example 8

### Western Blotting (2)

In this example, in the same manner as in Example 4, the action mechanism for killing cells by Pro-IP6 was checked by Wester blotting using a Jurkat cell and various antibodies.

Jurkat cells (1×10⁵ cells/well, culture fluid: 500 μL) were seeded in a 24-well plate to be cultured overnight. Thereafter, DMSO (1%, Fujifilm Wako Pure Chemical Corporation), an aqueous solution (1%) of IP6 (final concentration: 10 μM, Sigma Aldrich), or a DMSO solution (1%) of Pro-IP6 (final concentration: 1 μM or 10 μM) was added thereto, followed by culturing for 8 hours or 24 hours. A supernatant was removed, the resultant was washed with 1×PBS (−) once, Laemmli sample buffer was added thereto, and the resultant was boiled at 100° C. for 1 hour to dissolve the cells. The resultant was subjected to electrophoresis and then transferred onto a membrane (Millipore).

The resultant was reacted with, as a primary antibody, an anti-TRAF6 antibody (CST), an anti-pAMPK antibody (CST), or an anti-β-actin antibody (Sigma Aldrich). Detection was performed using ImmunoStar LD (Fujian Wako Pure Chemical Corporation).

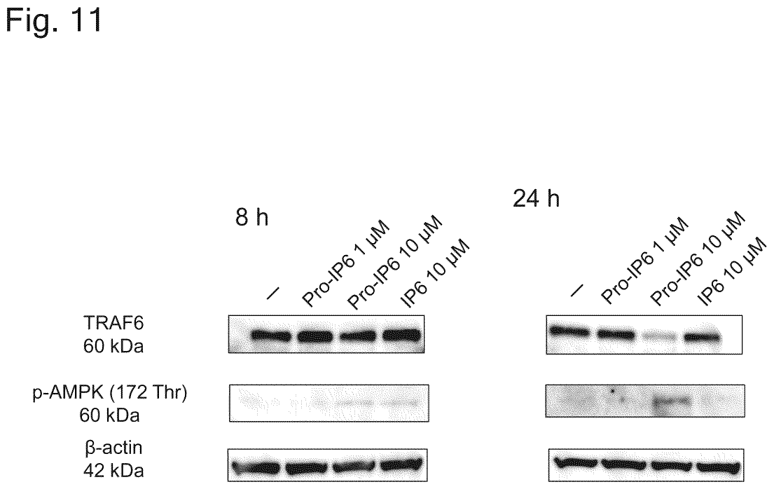

Results are illustrated in FIG. 11. It was observed that Pro-IP6 inhibits expression of TRAF6 leading to enhancement of survival of cancer cells, and activates AMPK leading to cancer suppression. The results obtained in this example suggest that IP6 is generated from Pro-IP6 in a Jurkat cell to exhibit the antitumor activity similar to that of IP6.

### Example 9

### Involvement of Pro-IP6 in Intracellular Aggregation of Virus Protein Gag

In this example, it was observed with a fluorescence microscope that Pro-IP6 is involved in aggregation of virus protein Gag in a cell.

HeLa cells (0.5×10⁵ cells/well, culture fluid: 300 μL) were seeded in a 8-well chamber slide (Thermo fisher scientific) to be cultured overnight. Lipofectamine 300 (Thermo fisher scientific) was used to introduce pUC or pEF-Gag(p17)cFLAG, pNL4-3/Gag-Venus, and the resultant was cultured for 10 hours. Thereafter, DMSO (1%, Fujifilm Wako Pure Chemical Corporation) or Pro-IP6 (final concentration: 10 μM) was added thereto, followed by culturing for another 4 hours. The thus obtained cells were fixed with 4% paraformaldehyde (Tokyo Chemical Industry Co., Ltd.), dyed with Hoechst 33342 (Thermo fisher scientific), and observed with a fluorescence microscope, BZ-X800 (Keyence).

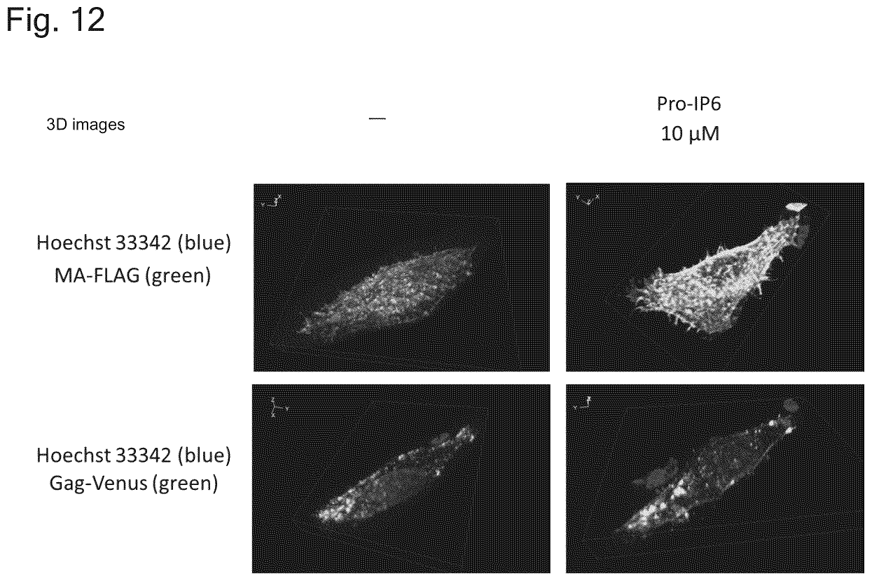

Photographs obtained as 3D images using the fluorescence microscope are illustrated in FIG. 12 (magnification×1000). In the photograph disposed at the lower right of FIG. 12, it was observed that virus protein Gag or MA region is aggregated in the Hela cells when Pro-IP6 is added.

### INDUSTRIAL APPLICABILITY

A compound of formula I is significantly incorporated into a cell, and exhibits, on a malignant cell such as a cancer cell, the same effects as those of phytic acid (IP6) including a cytotoxic effect, an antitumor effect and the like. Besides, Pro-IP6 is not toxic to a normal cell. A pharmaceutical composition and a cosmetic composition containing a compound of formula I are useful as a composition having higher effects than IP6 and sale for a living body.

1-12. (canceled)

13. A method for preventing or treating a disease, comprising:
administering to a target in need thereof an effective amount of a compound, or its pharmaceutically acceptable salt, of Formula I: 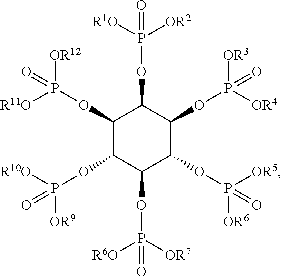
wherein all of R¹, R², R³, R⁴, R⁵, R⁶, R⁷, R⁸, R⁹, R¹⁰, R¹¹ and R¹² are butyryloxymethyl;
wherein the disease involves a condition selected from the group consisting of cell growth, cytotoxicity, weakened immune system, increase of cholesterol, kidney stone, cancer metastasis, and fibrosis.

14. The method according to claim 13, wherein the condition is selected from the group consisting of cancer, coronary artery disease, diabetes, and lithiasis.

15. The method according to claim 14, wherein the cancer is a cancer selected from the group consisting of leukemia, lymphoma, and myeloma.

16. The method according to claim 13, wherein the compound of Formula I is orally administered, transdermally administered, intraperitoneally administered, or intravenously administered.

17. The method according to claim 13, wherein the compound of formula I is hydrolyzed in a living body to change at least one of or all of R¹, R², R³, R⁴, R⁵, R⁶, R⁷, R⁸, R⁹, R¹⁰, R¹¹, and R¹² to H.

18. The method according to claim 13, wherein from about 0.01 mg/kg/day to about 2000 mg/kg/day of Formula I is administered to the target in need thereof

19. The method according to claim 13, wherein from about 1.0 mg/kg/day to about 200 mg/kg/day of Formula I is administered to the target in need thereof.

20. A method for imparting, to a target in need thereof, an activity selected from the group consisting of cell growth inhibition, cytotoxicity inhibition, enhancement of immune system, reduction of cholesterol, prevention of kidney stone, cancer metastasis inhibition, and fibrosis inhibition, the method including administering to the target in need thereof a compound, or its pharmaceutically acceptable salt, of formula I: 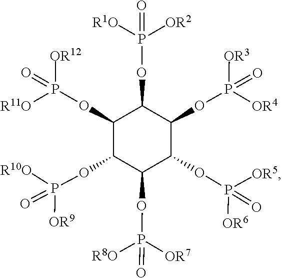
wherein all of R¹, R², R³, R⁴, R⁵, R⁶, R⁷, R⁸, R⁹, R¹⁰, R¹¹ and R¹² are butyryloxymethyl.

21. The method according to claim 20, wherein the compound of Formula I is orally administered, transdermally administered, intraperitoneally administered, or intravenously administered.

22. The method according to claim 20, wherein the compound of Formula I is hydrolyzed in a living body to change at least one of or all of R¹, R², R³, R⁴, R⁵, R⁶, R⁷, R⁸, R⁹, R¹⁰, R¹¹, and R¹² to H.

23. The method according to claim 20, wherein from about 0.01 mg/kg/day to about 2000 mg/kg/day of Formula I is administered to the target in need thereof.

24. The method according to claim 20, wherein from about 1.0 mg/kg/day to about 200 mg/kg/day of Formula I is administered to the target in need thereof.

25. The method according to claim 20, wherein administration of the compound of Formula I improves the activity by about 3% or more.

26. The method according to claim 20, wherein administration of the compound of Formula I improves the activity by about 10% or more.

27. The method according to claim 20, wherein administration of the compound of Formula I improves the activity by about 20% or more.
